(19) United States
(12) Patent Application Publication
(10) Pub. No.: US 2022/0362168 A1
(43) Pub. Date: Nov. 17, 2022

(54) ADMINISTRATION REGIMES OF CANNABINOIDS IN COMBINATION WITH CHEMOTHERAPEUTICS AGAINST CANCER

(71) Applicant: Jay Pharma Inc., Toronto (CA)

(72) Inventors: Wai Liu, London (GB); Angus Dalgleish, London (GB); Nadine Hall, London (GB)

(21) Appl. No.: 17/634,105

(22) PCT Filed: Aug. 9, 2019
(86) PCT No.: PCT/GB2019/052254

§ 371 Date: Feb. 9, 2022

(51) Int. Cl. A61K 31/05; A61K 31/704; A61K 31/337; A61K 31/7068; A61K 31/352; A61K 31/60; A61K 31/133; A61K 31/08; A61K 31/165; A61K 31/232; A61K 31/454; A61P 35/00; A61P 11/00

(52) U.S. Cl. CPC A61K 31/05; A61K 31/704; A61K 31/337; A61K 31/7068; A61K 31/352; A61K 31/60; A61K 31/133; A61K 31/08; A61K 31/165; A61K 31/232; A61K 31/454; A61P 35/00; A61P 11/00

(57) ABSTRACT

The present invention in the field of cancer therapeutics is based on the finding that when cannabinoids are administered to cancer subjects after a chemotherapeutic agent has been administered, the combined treatment leads to increased survival prognosis, a reduction in disease progression, stabilisation of disease state and inhibition of tumour growth than administration of the chemotherapeutic agent alone. There is provided a pharmaceutical composition comprising a chemotherapeutic agent for use in the treatment of bladder, brain and spinal cord, colorectal, head and neck, lung, lymphoma, neuroendocrine, oesophageal, ovarian, pancreatic and prostate cancer, wherein said treatment comprises a first phase in which the chemotherapeutic agent is administered, and a subsequent second phase in which a cannabinoid is administered.

### FIELD OF THE INVENTION

The invention relates to regimes of drug administration and drug combinations for use in the treatment of bladder, brain and spinal cord, colorectal, head and neck, lung, lymphoma, neuroendocrine, oesophageal, ovarian, pancreatic and prostate cancer.

### BACKGROUND TO THE INVENTION

Cancer is the second leading cause of death worldwide. Cancer can be caused by a number of factors including poor diet, obesity, smoking, lack of physical activity, tobacco use, alcohol use and infection. Cancer manifests as the unregulated over-proliferation of cells and can progress into metastasis, causing serious complications including death. The cellular changes which precede the development of cancer are the result of complex interactions between the genetics of a subject and the external factors the subject is exposed to, including physical, chemical and biological carcinogens.

Some types of cancer are susceptible to chemotherapeutic treatment whereby drugs are administered which disrupt over-proliferation of cells. However, because cancer cells contain largely the same proteins and other targets as the healthy cells in the body, there are few cancer-specific druggable targets, and chemotherapies often simply target all quickly proliferating cells in the body. As such, while these chemotherapies can be successful in suppressing tumours, there are side effects associated with them, and these become more severe the higher the dose of the chemotherapy.

The extracellular signal-regulated kinase (ERK) signalling pathway plays an important role in a large number of cellular processes such as proliferation and cell survival. It has emerged as the focal point for many signal transduction pathways through its modulation of numerous downstream substrates and targets such as those controlling cell cycle transit and gene transcription. One signalling protein important in this cascade is phosphorylated extracellular signal-regulated kinase (pERK). As tumours with high levels of this pERK tend to respond less favourably to treatment, it has been seen as a negative prognostic indicator for cancer progression. Interestingly, the levels of pERK can be raised in response to particular compounds. One such group of compounds which elevate pERK levels are known as phytocannabinoids. Phytocannabinoids also increase the expression of the cyclin-dependent kinase inhibitor p21^{2awf1}, the increased expression of which appears to be maintained by CBD, which inadvertently impedes cell death.

The phytocannabinoids are a group of chemicals extracted from the Cannabis plant. A number of phytocannabinoids are able to impede cancer cell growth, induce apoptosis and autophagy, and inhibit angiogenesis. The physiological effects of phytocannabinoids are thought to be partially mediated by binding to cannabinoid receptors which are expressed on a number of tissue types. The most widely known phytocannabinoid is Δ9-tetrahydrocannabinol (THC), and although it possesses these anticancer effects, it is also psychoactive. This presents various problems which have hampered clinical development of treatments using phytocannabinoids. Dosages have also traditionally been on the conservative side to negate the psychoactivity which would restrict the use of THC as a drug. Of the 80+ known phytocannabinoids, THC is likely to be the only phytocannabinoid which exhibit this psychoactivity.

There are an increasing number of reported studies which focus on examining the role of cannabinoids in in the management of cancer symptoms, particularly pain. In a disease such as cancer, the aetiology of which is complex, patients often feel that conventional therapies are not working for them and so they search for alternative medicines. It is then that they become aware of the use of Cannabis to treat cancer. The Cannabis products they use vary, and can be in the form of whole-plant extracts or purified oils; however, whatever the source, dosages are usually self-prescribed.

### SUMMARY OF THE INVENTION

It has been found by the present inventors that when cannabinoids are administered to subjects after a chemotherapeutic agent has been administered, the combined treatment leads to extended survival, a reduction in disease progression, stabilisation of disease state and inhibition of tumour growth than administration of the chemotherapeutic agent alone. As levels of pERK, a negative prognostic indicator for cancer, are increased by cannabinoids, such an effect is contrary to expectations.

According to a first aspect of the invention, there is provided a pharmaceutical composition comprising a chemotherapeutic agent for use in the treatment of a bladder, brain and spinal cord, colorectal, head and neck, lung, lymphoma, neuroendocrine, oesophageal, ovarian, pancreatic and prostate cancer, wherein said treatment comprises a first phase in which the chemotherapeutic agent is administered, and a subsequent second phase in which a cannabinoid is administered.

According to a second aspect of the invention, there is provided a pharmaceutical composition comprising a cannabinoid for use in the treatment of a bladder, brain and spinal cord, colorectal, head and neck, lung, lymphoma, neuroendocrine, oesophageal, ovarian, pancreatic and prostate cancer, wherein said treatment comprises a first phase in which a chemotherapeutic agent is administered, and a subsequent second phase in which the cannabinoid is administered.

According to a third aspect of the invention, there is provided a pharmaceutical composition comprising a cannabinoid for use in the treatment of a bladder, brain and spinal cord, colorectal, head and neck, lung, lymphoma, neuroendocrine, oesophageal, ovarian, pancreatic and prostate cancer in a subject, wherein the subject has previously been administered a chemotherapeutic agent.

According to a fourth aspect of the invention, there is provided a pharmaceutical composition comprising a chemotherapeutic agent and a cannabinoid for use in the treatment of a bladder, brain and spinal cord, colorectal, head and neck, lung, lymphoma, neuroendocrine, oesophageal, ovarian, pancreatic and prostate cancer, wherein the chemotherapeutic agent and cannabinoid are formulated or incorporated so as to facilitate sequential administration.

According to a fifth aspect of the invention, there is provided a method of treating bladder, brain and spinal cord, colorectal, head and neck, lung, lymphoma, neuroendocrine, oesophageal, ovarian, pancreatic and prostate cancer wherein said method comprises a first phase in which a chemotherapeutic agent is administered, and a subsequent second phase in which a cannabinoid is administered.

### BRIEF DESCRIPTION OF THE FIGURES

FIG. 1 plots the percentage viability of adenocarcinoma cells presented with differing levels of cannabidiol (CBD), as measured according to example 2.

FIG. 2 plots the relative levels of pERK in adenocarcinoma cells presented with differing levels of CBD, as measured according to example 3.

FIG. 3 plots the viability and live cell count relative to control of adenocarcinoma cells presented with varying concentrations of three representative chemotherapeutic agents, as measured according to example 4.

FIG. 4 plots the viability and live cell count relative to control of adenocarcinoma cells presented for two days with either CBD or one of the representative chemotherapeutic agents, then put into fresh media containing another of the compounds for a further two days. The compound presented in the first two days is before the dash, and the later compound is after the dash. CBD=Cannabidiol, GEM=Gemcitabine, DOX=Doxorubicin, PAC=Paclitaxel, as measured according to example 5.

### DETAILED DESCRIPTION OF THE INVENTION

The present inventors have found that when a cannabinoid is administered to a subject after administration of a chemotherapeutic agent, survival is extended, disease progression is reduced, disease state is stabilised and tumour growth is inhibited compared to the case in which just the chemotherapeutic agent is administered.

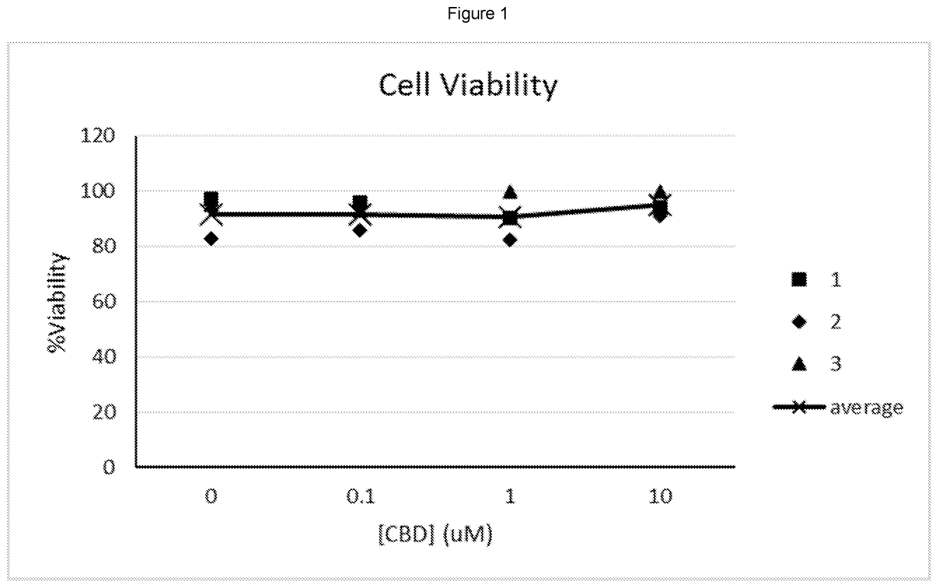

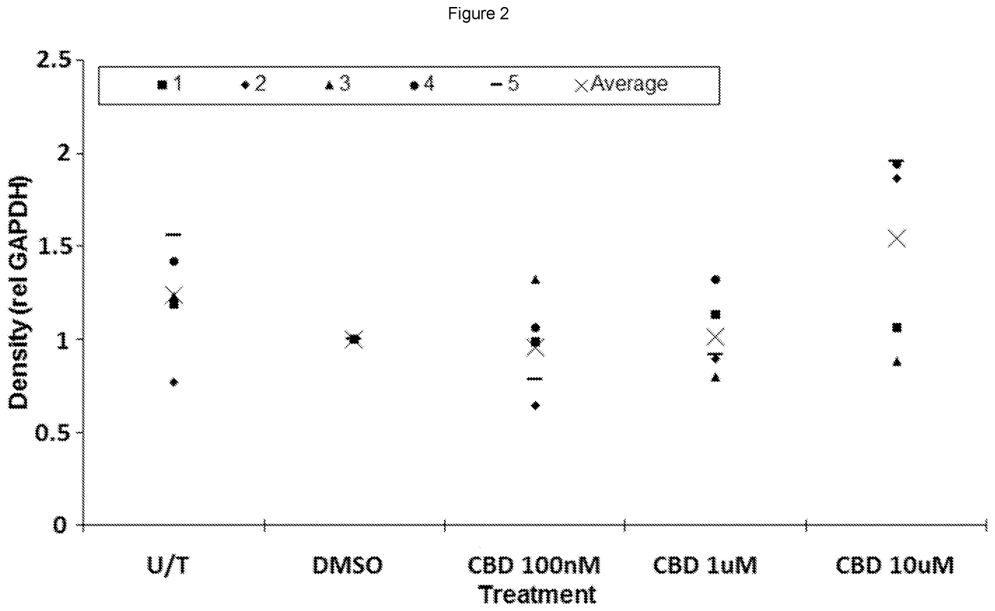

Cannabinoids have no effect on cancer cell viability in isolation, as seen in FIG. 1, and in fact even increase the concentration of pERK in adenocarcinoma cells, as seen in FIG. 2. The unexpected discovery that cannabinoids can be beneficial in cancer treatment is contrary to former understanding that markers such as pERK have been considered negative prognostic indicators for cancer.

Traditionally, cancer cells with high levels of pERK are seen as more likely to survive and proliferate than those with lower levels. Cannabinoids are known to increase levels of pERK and as such they would be expected to be poor cancer treatments. However, when administered as part of the current invention, cannabinoids increase the potency of the chemotherapeutic agent they follow, without being bound by theory.

As the chemotherapeutic agents of the current invention may, like most chemotherapy, cause unpleasant side-effects in patients, the ability to provide the same or greater therapeutic effect with a smaller overall dose of the chemotherapeutic agent minimises the risk and severity of side-effects in subjects.

Each of the aspects of the invention is aimed towards the treatment of bladder, brain and spinal cord, colorectal, head and neck, lung, lymphoma, neuroendocrine, oesophageal, ovarian, pancreatic and prostate cancer, and the treatment comprises two phases. In the first phase a chemotherapeutic agent is administered, and in the subsequent second phase a cannabinoid is administered.

As used herein, the terms “treating” and “treatment” and “to treat” refer to both therapeutic measures that cure, slow down, and/or halt progression of a diagnosed pathologic condition or disorder, and also to prophylactic or preventative measures that prevent and/or slow the development of a targeted pathologic condition or disorder. Therefore, those in need of treatment include those already with the disorder, those prone to have the disorder, and those in whom the disorder is to be prevented. In some instances, a subject is successfully “treated” for a tumour/cancer according to the present invention if the subject shows one or more of the following: a reduction in the number of, or complete absence of, cancer cells; a reduction in the tumour size; inhibition of, or an absence of, cancer cell infiltration into peripheral organs including, for example, the spread of cancer into soft tissue and bone; inhibition of, or an absence of, tumour metastasis; inhibition of, or an absence of, tumour growth; reduced morbidity and mortality; reduction in tumourigenicity, tumourigenic frequency, or tumourigenic capacity of a tumour; reduction in the number or frequency of cancer stem cells in a tumour; differentiation of tumourigenic cells to a non-tumourigenic state; or some combination of effects. Inasmuch as the treatment comprises two phases, the phases may be separated geographically and overseen by different health professionals.

As used herein, the term “survival” refers to the period of time a subject is alive.

Extended or increased survival is akin to an extended or increased life span, or the avoidance of death. The term “disease progression” refers to the physiological advancement of the cancer and the resulting physical effects upon the subject, such as the onset of cachexia. This term may also be used to represent an increase in the severity of the cancer, which may correlate to the grading of the cancer. Grading of the cancer may be any of stages 0-IV, wherein stage 0 represents that the cancer is in situ and has not spread, and stage IV indicates a secondary metastatic cancer. The grading of cancer severity is well known in the art.

The term “disease state” refers to the phenotypic characteristics of the cancer at cellular, physiological and whole subject levels. Disease state can be measured by a variety of methods such as microscopic analysis of tissue, assaying bodily fluids for cancer-related biomarkers and assessing a subjects physical symptoms. A stabilised disease state is when the cellular, physiological and a subjects physical symptoms have not increased or decreased in severity. The term “tumour growth” refers to the size, shape and weight of a solid tumour such as those observed in prostate cancer. Tumour growth may also refer to the level of angiogenesis, the invasive growth of a tumour into surrounding tissue or a change in cell morphology. The inhibition of tumour growth may be a reduction in any of these parameters.

The status of cancer disease in a subject may be assessed by a circulating tumour cell test and imaging techniques such as MRI and PET.

As used herein, the term “tumour/cancer” refers to any mass of tissue, however small, that results from excessive cell growth, proliferation and/or survival, either benign (noncancerous) or malignant (cancerous), including pre-cancerous lesions. Any form of bladder, brain and spinal cord, colorectal, head and neck, lung, lymphoma, neuroendocrine, oesophageal, ovarian, pancreatic and prostate cancer is envisaged.

As used herein, “bladder, brain and spinal cord, colorectal, head and neck, lung, lymphoma, neuroendocrine, oesophageal, ovarian, pancreatic and prostate cancer” refers to any mass of tissue that results from excessive cell growth, proliferation and/or survival of these tissues. Bladder, brain and spinal cord, colorectal, head and neck, lung, lymphoma, neuroendocrine, oesophageal, ovarian, pancreatic and prostate cancer may also refer to other pre-cancerous conditions, such as general cell hyperplasia in these tissues.

The term “bladder cancer” may refer to transitional cell carcinoma, squamous cell carcinoma, adenocarcinoma, sarcoma and small cell carcinoma of the bladder.

The term “brain and spinal cord cancer” may refer to cancers which cause extradural, intradural, intramedullary and extramedullary tumours. For example, the cancer of the spinal cord and brain can be meningioma, pituitary adenoma, medulloblastoma, oligodendroglioma, schwannoma, ependymoma, haemangioblastoma, astrocytoma, neuroblastoma, Ewing's sarcoma, pineocytoma, subependymoma, tanycytic ependymoma and choroid plexus tumours. Preferably the brain and spinal cord cancer is a glioma, most preferably anaplastic ependymoma, diffuse intrinsic pontine glioma (DIPG) and glioblastoma multiforme.

The term “colorectal cancer” may refer to adenocarcinoma, gastrointestinal stromal tumours, carcinoids, Turcot syndrome, Peutz-Jeghers syndrome, familial colorectal cancer and juvenile polyposis coli.

The term “head and neck cancer” refers to cancers which develop in the mouth, throat, sinuses, salivary glands, external auditory canal, middle ear, inner ear, nasopharynx, larynx, oropharynx, hypopharynx, lymph nodes of the neck. These cancers can be squamous-cell carcinoma or adenocarcinoma.

The term “lung cancer” refers to mesothelioma, pancoast tumours, small cell lung cancer and non small cell lung cancer. Non small cell lung cancer can be adenocarcinoma, squamous cell cancer, large cell carcinoma and undifferentiated non small cell lung cancer. Preferably the lung cancer is non small cell lung cancer.

The term “lymphoma cancer” refers to Non-Hodgkin's lymphoma, B-cell lymphoma, diffuse large B-cell lymphoma, T-cell lymphoma, Burkitt's lymphoma, follicular lymphoma, mantle cell lymphoma, primary mediastinal B cell lymphoma, small lymphocytic lymphoma, waldenstrom macroglobulinemia (lymphoplasmacytic lymphoma), Hodgkin's lymphoma, lymphocyte-depleted

Hodgkin's disease, lymphocyte-rich Hodgkin's disease, mixed cellularity Hodgkin's lymphoma, nodular lymphocyte-predominant Hodgkin's disease, nodular sclerosis Hodgkin's lymphoma. Preferably the lymphoma cancer is non-Hodgkin's lymphoma.

The term “neuroendocrine cancer” refers to cancers originating from endocrine cells or endocrine gland cells in any tissue, including any endocrine tissues affected by the cancer types disclosed here. Neuroendocrine cancers originating from breast tissue are not supported here.

The term “oesophageal cancer” refers to squamous cell carcinoma and adenocarcinoma.

The term “ovarian cancer” refers to ovarian epithelial cancer, germ cell tumours, sex cord-stromal tumours, ovarian sarcoma, Krukenberg tumours and ovarian cysts.

The term “pancreatic cancer” refers to pancreatic exocrine and endocrine cancers. Specifically, exocrine pancreatic cancer can be acinar cell carcinoma, adenosquamous carcinoma, colloid carcinoma, giant cell tumor, hepatoid carcinoma, mucinous cystic neoplasms, pancreatoblastoma, serous cystadenoma, signet ring cell carcinoma, solid and pseudopapillary tumors, squamous cell carcinoma, and undifferentiated carcinoma. Endocrine pancreatic cancer can be insulinoma, glucagonoma, gastrinoma, somatostatinoma, vasoactive intestinal peptideomas and pancreatic polypeptideomas.

The term “prostate cancer” refers to acinar adenocarcinoma, ductal adenocarcinoma, transitional cell (or urothelial) cancer, squamous cell cancer, small cell prostate cancer, carcinoid and sarcoma.

All of these cancer types can be high grade (fast growing) or low grade (slow growing).

The term “invasive cancer” refers to cancer that has spread beyond the primary tumour in which it developed, and is growing in surrounding, healthy tissues. Invasive cancer is sometimes referred to as infiltrating cancer. The term is intended to include all primary invasive cancers.

The cancer may be at any stage of progression. In any stage, the cancer may be relapsed or recurrent cancer, in which the cancer returns after a period of improvement or remission.

Cancer treatments are often particularly effective on subsets of tumours expressing particular biomarkers, and the cancer to be treated by the current invention may be characterised by its expression of various biomarkers. The subject may have a tumour which has been clinically tested and shown to be positive or negative for clinically relevant levels of one or more biomarkers selected from any standard biomarker known in the art. For example, the biomarker could be CA-125 which is elevated in ovarian cancer, PSA which is elevated in prostate cancer, or KRAS which is elevated in colon cancer.

As used herein, “clinically relevant levels” refer to expression levels of a given gene which may serve to characterise the cancer as belonging to a particular clinical category. Different clinical categories of cancer exhibit different molecular abnormalities, and may be most effectively treated in differing ways, as such the ability to accurately categorise tumours increases the efficacy of treatment.

When cancer is in a subject, the subject may be any animal susceptible to the pathology, including but not limited to humans, non-human primates, horses, canines, felines, and rodents.

In the first phase of the treatment, a chemotherapeutic agent is administered to the subject having cancer. The chemotherapeutic agent may be any chemotherapeutic agent, such as an anti-metabolite agent, growth factor inhibitor, aromatase inhibitor, a platinum-based agent, a PARP inhibitor or a checkpoint inhibitors. Preferably, the chemotherapeutic agent is an anthracycline, a taxane, or a nucleoside analogue. The agent may be any pharmaceutically acceptable salt, or prodrug of the above, and administration may be via any acceptable route, potentially chosen from such routes as the oral, buccal, sublingual, nasal, pulmonary, intravenous, intraperitoneal, intramuscular, rectal, vaginal, topical, intraocular and/or transdermal routes. Any combination of the above chemotherapeutic agents is also envisaged. Any vehicle including albumin and castor oil is envisaged for each of the chemotherapeutic agents.

Also envisaged is any formulation or administration device which incorporates a chemotherapeutic agent and a cannabinoid in such a way that the two are sequentially released as per the invention. This may be, for example, a pill or other formulation with two differentially structured compartments, or the two agents pre-incorporated into two different materials with different release rates, it may be an implanted device with differentially situated compartments or drug-soaked areas, or it may be a device which is electronically programmed to inject each at particular intervals.

The dosage regime of the chemotherapeutic agent is not particularly limited, but preferable regimens are envisaged for different chemotherapeutic agents.

Preferably the chemotherapeutic agent of the current invention is an anthracycline, a taxane, or a nucleoside analogue. Where ranges are given herefrom, they may be read as approximate, and are inclusive of the range boundaries.

For anthracyclines administration may be such that the concentration within the patient is generally within the range of 0.1-10 nM, preferably within the range of 0.5 nM-5 nM, more preferably within the range of 0.8-2 nM, or it may be such that the concentration within the patient is generally within the range of 10 nM-1 μM, preferably within the range of 50-500 nM, more preferably within the range of 80-200 nM. The anthracycline may be administered at any physiologically acceptable dose, preferably at a dose of 10-90 mg/m², more preferably 30-70 mg/m², yet more preferably 40-60 mg/m², most preferably 45-55 mg/m².

Anthracyclines may be administered at any physiologically allowable frequency. For example an anthracycline may be administered intravenously once every three weeks, once every two weeks, once a week, once every other day, or once a day. Preferably anthracyclines are administered once a week, more preferably once every three weeks.

The anthracycline may be any, for example daunorubicin, doxorubicin, epirubicin, idarubicin, valrubicin, nemorubicin, pixantrone, or sabarubicin, and is preferably doxorubicin.

For taxanes, administration may be at any dose but is preferably such that the concentration within the patient is generally within the range of 0.1-10 nM, preferably within the range of 0.5-5 nM, more preferably within the range of 0.8-2 nM, or it may be such that the concentration within the patient is generally within the range of 10 nM-1 μM, preferably within the range of 50-500 nM, more preferably within the range of 80-200 nM. Taxanes may be administered at any physiologically acceptable dose, preferably at 50-260 mg/m², more preferably 100-210 mg/m², even more preferably 125-185 mg/m², yet more preferably 135-175 mg/m², most preferably 150-160 mg/m². Taxanes may be administered at any physiologically allowable frequency. For example a taxane may be administered once every three weeks, once every two weeks, once a week, once every other day, or once a day.

The taxane may be any, for example, paclitaxel, nab-paclitaxel, CrEL-paclitaxel, paclitaxel poliglumex, cationic liposomal paclitaxel, polymeric-micellar paclitaxel, DHA-paclitaxel, docetaxel, cabazitaxel, abraxane, DJ-927, BMS-184476, larotaxel, taxotere, hongdoushan A, B, or C, and is preferably paclitaxel.

As used herein, “nucleoside analogue” refers to a chemotherapeutic agent having a mechanism of action which relies on the agent, or a downstream metabolite of the agent, substituting for a nucleoside or nucleotide in a biochemical reaction, or otherwise disrupting the addition of a nucleoside or nucleotide into a polynucleotide chain. Agents which are analogues of nucleic acid precursors are also envisaged. As such, it will be appreciated that nucleotide analogues as well as precursor analogues also form part of the group of nucleoside analogues.

For nucleoside analogues administration is preferably such that the concentration within the patient is generally within the range of 1-100 μM, preferably 5-50 μM, more preferably 8-20 μM. Nucleoside analogues may be administered at any physiologically allowable dose but are preferably administered at a dose of 250-2,250 mg/m², more preferably 500-2,000 mg/m², even more preferably 800-1,700 mg/m², yet more preferably 1,100-1,400 mg/m², most preferably 1,200-1,300 mg/m². They may be administered at any physiologically allowable frequency, and are preferably administered once a week, more preferably once every three weeks.

The nucleoside analogue may be any encompassed compound, for example, gemcitabine, azacitidine, azathioprine, capecitabine, doxifluridine, fluorouracil, hydroxyurea, mercaptopurine, methotrexate, tioguanine, DHAC, zebularine, cytosine arabinoside. Preferably the nucleoside analogue is gemcitabine.

In a medical setting, it is envisaged that the dose and administration frequency of the chemotherapeutic agent may be adjusted on a case-by-case basis depending on the status and needs of the patient, and the progress of the treatment up to the point of administration.

In the subsequent second phase of the treatment regimen, a cannabinoid is administered.

Cannabinoids are a class of compounds understood by the skilled person which comprise those abundantly made by plants of the Cannabis genus, as well as endocannabinoids which are synthesised in animals. Synthetic compounds which are structurally similar to natural cannabinoids and/or are active against cannabinoid receptors are also envisaged.

Cannabinoids exert various effects on the physiology of mammalian and tumour cells, and many of these effects are mediated by two G protein-coupled receptors known as cannabinoid receptors and deemed CB1 and CB2. These receptors are known to interact with at least five structurally distinct classes of compounds. These include the plant-derived classical cannabinoids, such as tetrahydrocannabinol and cannabinol; the non-classical bicyclic cannabinoid agonists, such as CP55,940; the endogenous cannabinoid agonists, such as anandamide (AEA); the aminoalkylindole (AAI) agonists, such as WIN55,212-2; and the antagonist/inverse agonists, such as SR141716A (Pertwee, 1995). Each class of compound is envisaged within the term cannabinoid, and each class may be useful for treating different aspects of bladder, brain and spinal cord, colorectal, head and neck, lung, lymphoma, neuroendocrine, oesophageal, ovarian, pancreatic and prostate cancer.

As the compounds can be agonistic or antagonistic, and some are more active against CB1, while others more active against CB2, compounds may be directed towards treatment of specific stages and categories of bladder, brain and spinal cord, colorectal, head and neck, lung, lymphoma, neuroendocrine, oesophageal, ovarian, pancreatic and prostate cancer. For example, some may bind specifically to CB2, such as JWH-133, and some may bind specifically to CB1, such as SR141716A. In certain pathologies, CB1 agonists may be desired, in others CB1 antagonists may be more appropriate. Alternatively or additionally, CB2 agonists and/or antagonists may be selected for use in the treatment of the current invention.

The cannabinoid of the current invention may be derived from a natural source, or be produced synthetically. The term “cannabidiol” (CBD) as used herein refers to a phytocannabinoid produced by Cannabis species. In some embodiments the cannabinoid used in the present invention is in a purified form, in a composition having at least 95% purity when the solvent is not counted. In other embodiments, the cannabinoid is a component of a plant extract, which may be administered to a subject. In some embodiments, a plant extract comprises, excluding the solvent, 10% to 95% cannabinoid. In some embodiments, a plant extract comprises 20% to 80% cannabinoid. In some embodiments, a plant extract comprises 30% to 70% cannabinoid. In some embodiments, a plant extract comprises 40% to 60% cannabinoid. The cannabinoid may be a botanical drug substance (BDS), defined in the Guidance for Industry Botanical Drug Products Draft Guidance, August 2000, US Department of Health and Human Services, Food and Drug Administration Centre for Drug Evaluation and Research as: “A drug derived from one or more plants, algae, or microscopic fungi. It is prepared from botanical raw materials by one or more of the following processes: pulverisation, decoction, expression, aqueous extraction, ethanolic extraction or other similar processes.” The cannabinoid is preferably cannabidiol (CBD).

The cannabinoid may be derived from a plant extract, and/or itself be a plant extract comprising one or more cannabinoid selected from one or more of the following categories of plant cannabinoids: Cannabigerol-type (CBG), cannabichromene-type (CBC), cannabidiol-type (CBD), cannabinodiol-type (CBND), tetrahydrocannabidiol-type (THC), cannabinol-type (CBN), cannabitriol-type (CBT), cannabielsoin-type (CBE), isocannabinoids, cannabicyclol-type (CBL), cannabicitran-type (CBT), cannabichromanone-type (CBCN).

The cannabinoid may also be a highly purified or chemically modified substance derived from natural sources. In some cannabinoids, for example CBD, it may comprise chemically modified derivatives of fully-decarboxylated cannabinoid which retain desired activity, or more preferably natural derivatives exhibiting improved activity which are produced according to standard principles of medicinal chemistry. In some embodiments, fully-decarboxylated CBD derivatives may exhibit a lesser degree of activity than the starting material so long as they retain sufficient activity to be therapeutically effective or exhibit improvements in properties desirable in pharmaceutically active agents such as improved solubility, enhanced uptake or reduced toxicity.

When used herein the term can refer to any cannabinoid, but the cannabinoid is preferably selected from the list containing cannabidiol, tetrahydrocannabinol, cannabidiolic acid, cannabinol, cannabigerol, cannabivarin, tetrahydrocannabivarin, cannabidivarin, cannabichromene, arachidonoylethanolamine, 2-arachidonoylglycerol, 2-arachidonoyl glyceryl ether, N-arachidonoyl dopamine, virodhamine, dronabinol, nabilone, rimonabant, anandamide, R-(+)-Met-anandamide, WIN-55,212-2, HU-210, JWH-133, SR144528, SR141716A, CP55,940 or combinations thereof, or pharmaceutically acceptable salts, solvates, hydrates, stereoisomers, clathrates, prodrugs, and analogues thereof which bring about equivalent effects.

In some embodiments, cannabidiol is administered alongside a tetrahydrocannabinol. In some embodiments, Sativex may be used as the source of tetrahydrocannabinol.

The cannabinoid may be administered at conventional amounts based on the particular cannabinoid and the details of the particular case. In certain embodiments, the cannabinoid is to be administered once or twice a day for three days, followed by three days of no cannabinoid treatment. Preferably this dosage schedule is repeated. In some embodiments, this dosage schedule is repeated without a break, such as in a cyclic fashion. In some embodiments, this dosage schedule is repeated with a break of a number of days, weeks or months.

Preferably, the cannabinoid is administered at a dose of between 1 mg and 600 mg, preferably between 5 mg and 500 mg, more preferably between 10 mg and 250 mg, most preferably between 20 mg and 125 mg. The cannabinoid may be administered at any frequency, for example, twice a day, three times a day, every other day, every third day, or every fourth day. Preferably the cannabinoid is administered twice daily. In a medical setting, it is envisaged that the dose and administration frequency may be adjusted on a case-by-case basis depending on the status and needs of the patient, and the progress of the treatment up to the point of latest administration. For example, twice daily dosages of 30 mg for larger tumours or more aggressive cancer may be utilised.

The method of administration is not particularly limited for cannabinoids, and they may be administered via the oral, buccal, sublingual, nasal, pulmonary, intravenous, intraperitoneal, intramuscular, rectal, vaginal, topical, intraocular and/or transdermal routes, preferably being administered sublingually. When the cannabinoid is administered via the pulmonary route, it may be via inhalation of a vapour comprising cannabinoids, optionally mediated by combustion or vaporisation of cannabinoid-comprising plant matter. If administered via the oral, buccal, sublingual or nasal route, this may be via an atomised spray.

This second phase of the treatment, in which the cannabinoid is administered, may last for any period of time, including indefinitely. It could alternatively last one month, two months or three months, but is preferably carried out for at least 6 months.

Once the second phase is complete, it is envisaged that in some circumstances the first phase will recommence, optionally followed again by the second phase, that is, numerous cycles of treatment may be necessary.

There may be a “recovery phase” in between the end of the first treatment phase and the start of the second treatment phase. During the recovery phase no chemotherapeutic agent or cannabinoid is administered. The recovery phase may be any length of time, may have a duration of, for example, a day, two days, or three days, and is preferably no more than a week. Also envisaged in the recovery phase is the situation in which a clinician decides to reduce the dose or administration frequency based on the status and needs of the patient, and the extent to which the treatment is being effective.

The subject may undergo radiotherapy following the second treatment phase of administration of the cannabinoid. It has been found that subjects may exhibit enhanced sensitivity to radiotherapy after undergoing the treatment schedule disclosed herein.

Also envisaged is a method of treating bladder, brain and spinal cord, colorectal, head and neck, lung, lymphoma, neuroendocrine, oesophageal, ovarian, pancreatic and prostate cancer wherein said method comprises a first phase in which a chemotherapeutic agent is administered, and a subsequent second phase in which a cannabinoid is administered.

Further envisaged is the use of a chemotherapeutic agent and a cannabinoid for the manufacture of a medicament for the treatment of bladder, brain and spinal cord, colorectal, head and neck, lung, lymphoma, neuroendocrine, oesophageal, ovarian, pancreatic and prostate cancer, wherein said chemotherapeutic agent is to be administered in a first phase, and said cannabinoid is to be administered in a subsequent second phase.

### EXAMPLES

### Example 1

The following example details the experiments and results disclosed in Kenyon, J., Liu, W., Dalgleish, A. (2018) Report of Objective Clinical Responses of Cancer Patients to Pharmaceutical-grade Synthetic Cannabidiol. Anticancer Research. 38: 5831-5835, which is herein incorporated by reference. This article by the present inventors was published in October 2018. To the extent that there is available a grace period, such that the article is not prior art, the content of the article supports the present application and the claims.

Patients were given synthetic, pharmaceutical-grade CBD (STI Pharmaceuticals), registered under the Pharmaceutical Specials scheme in oily drops at 5% (w/v) in 20 ml bottles. Each drop contained 1 mg of synthetic CBD in neutral oil. This was prescribed on an informed consent basis. 119 cancer patients decided to have this treatment (Table 1), and most of them had metastatic cancers. Of the 119 patients, 28 were given CBD as the only treatment. A third of these patients had already been taking Cannabis oil extracted from the Cannabis plant that had been bought on the Internet, with no beneficial response.

The majority of the patients were assessed using a circulating tumour cell test before and after treatment, since this is cheaper than carrying out repeated scans. A number of patients however, as a matter of a normal treatment course, had relevant scans.

CBD was administered on a three days on and three days off basis, which clinically was found to be more effective than giving it as a continuous dose. The average dose was 10 mg twice a day. For increased tumour mass, the dose was increased, in some cases up to 30 drops twice a day (30 mg). In a number of cases where stable disease was present, the dose was reduced to five drops twice a day (5 mg). In some cases, Sativex, which is licensed for use in multiple sclerosis, was used in conjunction with CBD as a source of THC, which synergises with CBD. A fraction of the dose used for multiple sclerosis was used. Two sprays of Sativex were given twice a day in three days on and three days off pattern, as in the case of pharmaceutical-grade synthetic CBD; patients on continuous dosing did not do as well as those on this on-off repeating regimen. Some of the patients reverted to Cannabis oil bought on the Internet, and following this, 80% of these cases relapsed.

A maximum tolerated dose for CBD was unable to be defined, as there was a complete absence of side effects. The minimum duration of treatment required for CBD was six months, but many continued for longer. Less than six months appeared inadequate and had little effect, and therefore cases in which CBD was used for less than six months have been defined as un-assessable, and not included in the current cohort of 119 cases.

Clear objective evidence of potential efficacy was sought where no other treatment option was available. The most impressive case was a five-year-old male patient with an anaplastic ependymoma, a very rare brain tumour. The patient had had all standard treatments, surgery on two occasions followed by chemotherapy and conformal photon radiotherapy. No further treatment options were available to him when treatment on CBD started. A scan carried out after treatment of ten months showed that tumour volume had decreased by ˜60%.

Subsequent scans continued to show disease stability. CBD was the only treatment.

Another impressive case was a 50-year-old patient with progressive tanycytic ependymoma Grade 2, treated with biopsy and radical radiotherapy for two years. He refused chemotherapy, and had no further treatment options. He started on pharmaceutical-grade synthetic CBD a year after radiotherapy ended, at a dose of 10 drops twice a day, three days on and three days off (10 mg). Prior to this he had been taking, for some time, metformin, mebendazole, doxycycline and atorvastatin from an oncology clinic in Central London.

A scan six months later showed tumour reduction. At that point the patient stopped taking pharmaceutical-grade synthetic CBD and switched to Cannabis oil extract obtained from an internet website. Further scans carried out a year later showed doubling of tumour size and more growth down the brain stem. He has since restarted pharmaceutical-grade synthetic CBD and throughout continued to take the metformin, atorvastatin, doxycycline and mebendazole. So, the only change had been stopping the pharmaceutical-grade synthetic CBD and switching to Cannabis oil extract obtained on the Internet.

Other patients who clearly improved using pharmaceutical grade synthetic CBD had prostate cancer, breast cancer, oesophageal cancer and a lymphoma, and these are summarised in Table 2 below.

TABLE 1
Tabular presentation of our results on 119 cancer patients.

| | | | Extended | | | | CBD | | |
|---|---|---|---|---|---|---|---|---|---|
| | Tumour | Stable | median | Slowed | No | | as only | Unknown | Total |
| Cancer type | free | disease | survival | progression | effect/result | Died | treatment | outcome | cases |
| Anaplastic ependymoma | | | 3 | | | | 3 | | 3 |
| DIPG | | | 1 | | | | 1 | | 1 |
| Glioblastoma multiforme | | | 4 | | | 3 | 4 | | 7 |
| Bladder | | 1 | 1 | | | | | | 2 |
| Breast | 7 | 21 | 8 | 3 | 3 | 6 | 6 | | 39 |
| Head and Neck | 1 | | | | | | | | 2 |
| Prostate | | 10 | 3 | | | 3 | 6 | | 16 |
| Neuroendocrine | | 1 | | | | | | | 1 |
| Non-Hodgkin’s lymphoma | 1 | 6 | | | | 1 | 3 | | 8 |
| Non-small cell lung | | | 2 | | | 2 | 2 | | 2 |
| Colorectal | 1 | | 9 | 1 | 1 | 6 | | | 13 |
| Pancreatic | | | 2 | | | 2 | 2 | | 4 |
| Ovarian | | | 5 | 1 | | 3 | 1 | | 6 |
| Miscellaneous | 2 | 6 | 5 | 1 | 1 | 1 | | 1 | 15 |
| Total | 12 | 45 | 43 | 8 | 5 | 27 | 28 | 1 | 119 |

TABLE 2
Examples of patients who have been using pharmaceutical grade synthetic CBD

| Age/Gender | Diagnosis | Comments |
|---|---|---|
| 72/male | Prostate cancer | Patient has had cancer immunotherapy, sono and photodynamic therapy which was successful. On resumption of |
| | | testosterone injections his prostate specific antigen (PSA) levels increased to 16. We started him on CBD at a dose |
| | | of 10 drops twice a day (10 mg), three days on and three days off. There was a reduction in circulating tumour cells |
| | | (CTCs) with CBD alone from an initial 8.1 cells/7.5 ml to 5.9 cells/7.5 ml, then a steady reduction over the course of |
| | | 12 months of 4.8, 4.2 then 3.2 cells/7.5 ml. He is still under treatment. |
| 68/female | Breast cancer | Patient was diagnosed with progressive disease. She started local radiotherapy. We started her on CBD, all |
| | with bone | subsequent scans showed stable disease. She has had no treatment other than CBD following radiotherapy. |
| | metastases | |
| 65/female | Oesophageal | Patient was diagnosed. She had a stent put on place at that time and was given an expected survival of three |
| | cancer | months. Since then, she has been on CBD as the only treatment, and she has continued to refuse all standard |
| | | treatments and investigations. We last saw her six months later when she was looking well and had in fact regained |
| | | weight. She died 14 months later. |
| 65/female | Breast cancer | Patient was diagnosed and refused all conventional treatments and investigations. On examination she had a large |
| | | fungating lesion 15 cm in diameter in the left breast, and also palpable left axillary nodes. She began treatment with |
| | | CBD. We persuaded her to have radiotherapy a month later. She only agreed to have half the recommended |
| | | treatment course. She has continued on CBD alone and on her last appointment the tumour in her left breast was 2 |
| | | cm in diameter, with no palpable axillary nodes. |
| 62/female | Breast cancer | We first saw this patient and she had been on CBD as the only treatment at that time. We carried out various CTC |
| | | tests which showed 10.6 cells per 7.5 ml. Subsequent tests 9 months later, 12 months later, two years later and |
| | | three years later showed CTCs to be 7.3, 6.8, 5.0 and 3.9 cells/7.5 ml, respectively. Patient is currently stable with |
| | | no symptoms. |
| 67/female | Lobular breast | Patient was diagnosed. We first saw her 16 months later. We gave her CBD 7 months later, which is the only |
| | cancer | method of treatment. Initial CTCs were 9.3 cells per 7.5 ml. Follow-up measurements 12 months later, 18 months |
| | | later and 30 months later have been 7.5, 6.8 and 3.0 cells/7.5 ml, respectively. All standard clinical investigations |
| | | and scans have been normal since 12 months after initial administration of CBD. |

### Example 2

An adenocarcinoma cancer cell line, growing exponentially, was reset at a concentration of 1×10⁴/ml and allowed to adhere overnight. Cannabidiol (CBD) was added at 0.1, 1, or 10 μM, and cell viability assessed after 48 h by cell counting, using the dye trypan blue to discriminate dead from live cells, the results of which are displayed in FIG. 1.

As the viability of the cancer cells was similarly high at all administered concentrations, including 0, the evidence suggests that the viability of cancer cells is unaffected by CBD in this range. As such, it appears that cannabinoids do not have a direct cytotoxic effect on cancer cells. Therefore if a cannabinoid is used in conjunction with another agent, and an effect on cancer viability is seen which is different to the effects of the other agent alone, then without being bound by theory these effects must stem from some form of mechanistic interaction between the cannabinoid and the other agent.

### Example 3

An adenocarcinoma cell line, growing exponentially, was reset at a concentration of 1×10⁴/ml and allowed to adhere overnight. Cannabidiol (CBD) was added at 100 nM, 1 μM, or 10 μM, and cells were harvested for standard western blotting techniques after 48 h. Immunoprobing was performed using anti-pERK antibodies, and the densities of each band determined. These values were then expressed relative to a GAPDH loading control. The data are displayed in FIG. 2, and show the individual data points for 5 separate experiments.

As can be seen, relative to the controls, pERK was increased in cells exposed to higher levels of CBD. As pERK is traditionally seen as a negative prognostic indicator for cancer progression, one would predict from this data that CBD would increase the viability and proliferation of cancer cells.

### Example 4

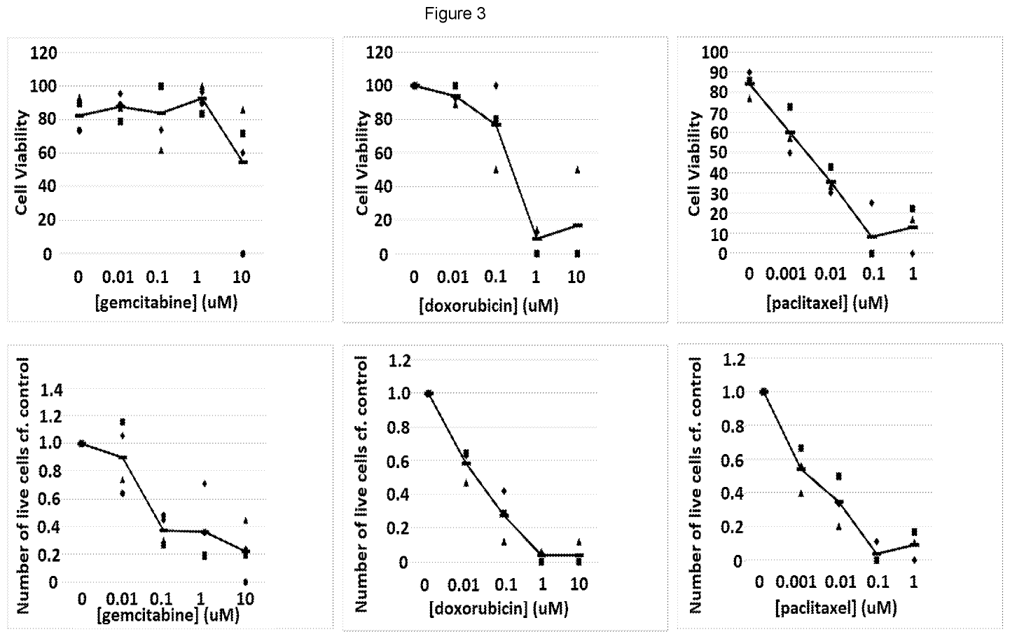

An adenocarcinoma cell line, growing exponentially, was reset at a concentration of 1×10⁴/ml and allowed to adhere overnight. Either gemcitabine, doxorubicin or paclitaxel was added, at 0.01, 0.1, 1, or 10 μM for gemcitabine and doxorubicin, and 0.001, 0.01, 0.1, or 1 μM for paclitaxel. Cell viability was assessed after 48 h by cell counting, using the dye trypan blue to discriminate dead from live cells. The data are displayed in FIG. 3, and confirm that the concentrations used in example 5 are appropriate.

### Example 5

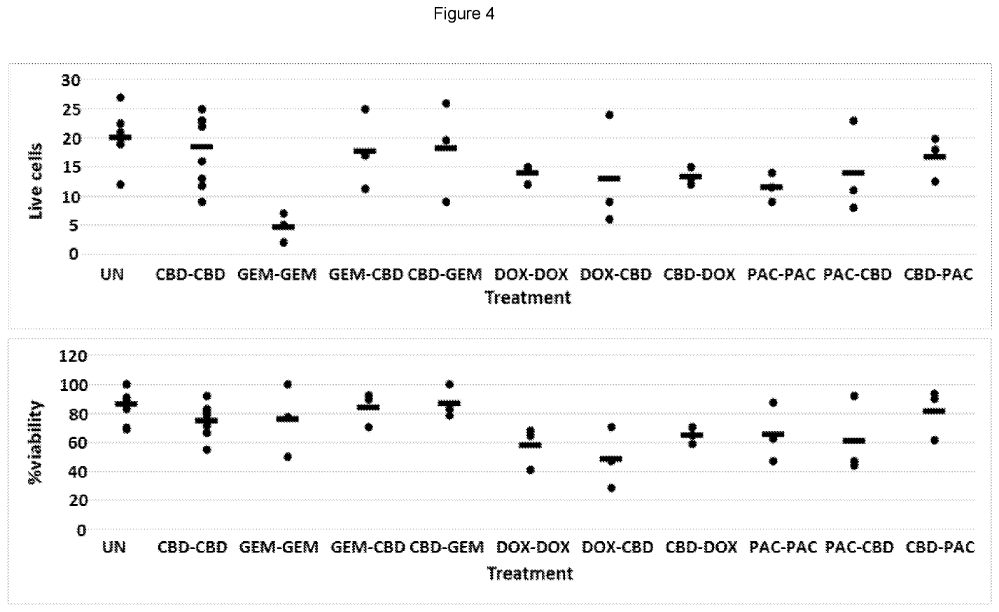

An adenocarcinoma cell line, growing exponentially, was reset at a concentration of 1×10⁴/ml and allowed to adhere overnight. A treatment schedule was then employed that involved two rounds of treatment each lasting 48 h, meaning a complete treatment schedule would be over 96h. For the first round of treatment, either CBD (10 μM), gemcitabine (GEM: 10 μM), doxorubicin (DOX: 100 nM) or paclitaxel (PAC: 1 nM) was added to the cells and they were left to grow for 48 h. After this time, exhausted media was removed and replaced with fresh medium supplemented with any of the drugs as listed earlier. After a further 48 h, cell viability was assessed by cell counting, using the dye trypan blue to discriminate dead from live cells. The data are displayed in FIG. 4.

The data reveal a number of surprising trends. Firstly, the order of administration of the chemotherapeutic agent and cannabinoid has a notable effect on the viability and live cell count of the treated cancer cells. In all cases, the cancer treatment administering a chemotherapeutic agent as a first agent, followed by a cannabinoid, delivers similar or superior results to the case in which they are administered the other way around.

A further surprising result is that in many cases, treatment with a chemotherapeutic agent followed by a cannabinoid offers superior results to double treatment with a chemotherapeutic agent, even though such a treatment amounts to administering just half the dose of chemotherapeutic agent over the course of the experiment. Given that CBD was shown in FIG. 1 to be inactive in treating cancer in isolation, and is known to produce fewer unpleasant side-effects than the chemotherapy agents tested herein, such a treatment can be expected to provide similar or superior cancer-suppressing results to chemotherapeutic agents alone, with less severe side effects.

1. A pharmaceutical composition comprising a cannabinoid for use in the treatment of a bladder, brain and spinal cord, colorectal, head and neck, lung, lymphoma, neuroendocrine, oesophageal, ovarian, pancreatic and prostate cancer, wherein said treatment comprises a first phase in which a chemotherapeutic agent is administered, and a subsequent second phase in which the cannabinoid is administered.

2. (canceled)

3. (canceled)

4. The pharmaceutical composition for use according to claim 1 wherein the chemotherapeutic agent and cannabinoid are formulated or incorporated so as to facilitate sequential administration.

5. The pharmaceutical composition for use according to claim 1, wherein the brain and spinal cord cancer is anaplastic ependymoma, diffuse intrinsic pontine glioma or glioblastoma multiforme, the lung cancer is non small cell lung cancer, and the lymphoma cancer is non-Hodgkin's lymphoma.

6. The pharmaceutical composition for use according to claim 1 wherein the chemotherapeutic agent is selected from the list consisting of anthracyclines, taxanes, and nucleoside analogues, and pharmaceutically acceptable salts and prodrugs thereof, wherein:
i) the chemotherapeutic agent is doxorubicin, wherein the doxorubicin is administered to a subject at a dose of 40-60 mg/m²;
ii) the chemotherapeutic agent is paclitaxel, wherein the paclitaxel is administered to a subject at a dose of 135-175 mg/m²; or
iii) the chemotherapeutic agent is gemcitabine, wherein the gemcitabine is administered to a subject at a dose of 1,100-1,400 mg/m².

7. The pharmaceutical composition for use according to claim 1, wherein the cannabinoid is selected from the list consisting of cannabidiol, tetrahydrocannabinol, cannabidiolic acid, cannabinol, cannabigerol, cannabivarin, tetrahydrocannabivarin, cannabidivarin, cannabichromene, arachidonoylethanolamine, 2-arachidonoylglycerol, 2-arachidonoyl glyceryl ether, N-arachidonoyl dopamine, virodhamine, dronabinol, nabilone, rimonabant, anandamide, R-(+)-Met-anandamide, WIN-55,212-2, HU-210, JWH-133, SR144528, SR141716A, CP55,940, or combinations thereof.

8. The pharmaceutical composition for use according to claim 1, wherein the cannabinoid is cannabidiol and is administered alongside a tetrahydrocannabinol.

9. The pharmaceutical composition for use according to claim 1, wherein at least one of:
administration of the chemotherapeutic agent is to be carried out once every three weeks or once a week,
administration of the cannabinoid is to be carried out twice a day for a period of from 1 to 7 days; or
administration of the cannabinoid is to be carried out after a recovery phase lasting from 1 to 7 days, during which neither the chemotherapeutic agent nor the cannabinoid is administered.

10. (canceled)

11. (canceled)

12. The pharmaceutical composition for use according to claim 9, wherein cyclic administration of the cannabinoid is to be undertaken for at least 6 months.

13. The pharmaceutical composition for use according to claim 1, wherein administration of the cannabinoid is sublingual.

14. A method of treating bladder, brain and spinal cord, colorectal, head and neck, lung, lymphoma, neuroendocrine, oesophageal, ovarian, pancreatic and prostate cancer wherein said method comprises a first phase in which a chemotherapeutic agent is administered, and a subsequent second phase in which a cannabinoid is administered.

15. A method of treatment according to claim 14, wherein the subject undergoes radiotherapy following administration of the cannabinoid.

16. A pharmaceutical composition comprising a chemotherapeutic agent for use in the treatment of a bladder, brain and spinal cord, colorectal, head and neck, lung, lymphoma, neuroendocrine, oesophageal, ovarian, pancreatic and prostate cancer, wherein said treatment comprises a first phase in which the chemotherapeutic agent is administered, and a subsequent second phase in which a cannabinoid is administered.

17. The pharmaceutical composition for use according to claim 16, wherein the chemotherapeutic agent and cannabinoid are formulated or incorporated so as to facilitate sequential administration.

18. The pharmaceutical composition for use according to claim 16, wherein the brain and spinal cord cancer is anaplastic ependymoma, diffuse intrinsic pontine glioma or glioblastoma multiforme, the lung cancer is non small cell lung cancer, and the lymphoma cancer is non-Hodgkin's lymphoma.

19. The pharmaceutical composition for use according to claim 16, wherein the chemotherapeutic agent is selected from the list consisting of anthracyclines, taxanes, and nucleoside analogues, and pharmaceutically acceptable salts and prodrugs thereof, wherein:
i) the chemotherapeutic agent is doxorubicin, wherein the doxorubicin is administered to a subject at a dose of 40-60 mg/m²;
ii) the chemotherapeutic agent is paclitaxel, wherein the paclitaxel is administered to a subject at a dose of 135-175 mg/m²; or
iii) the chemotherapeutic agent is gemcitabine, wherein the gemcitabine is administered to a subject at a dose of 1,100-1,400 mg/m².

20. The pharmaceutical composition for use according to claim 16, wherein the cannabinoid is selected from the list consisting of cannabidiol, tetrahydrocannabinol, cannabidiolic acid, cannabinol, cannabigerol, cannabivarin, tetrahydrocannabivarin, cannabidivarin, cannabichromene, arachidonoylethanolamine, 2-arachidonoylglycerol, 2-arachidonoyl glyceryl ether, N-arachidonoyl dopamine, virodhamine, dronabinol, nabilone, rimonabant, anandamide, R-(+)-Met-anandamide, WIN-55,212-2, HU-210, JWH-133, SR144528, SR141716A, CP55,940, or combinations thereof.

21. The pharmaceutical composition for use according to claim 16, wherein the cannabinoid is cannabidiol and is administered alongside a tetrahydrocannabinol.

22. The pharmaceutical composition for use according to claim 16, wherein at least one of:
administration of the chemotherapeutic agent is to be carried out once every three weeks or once a week;
administration of the cannabinoid is to be carried out twice a day for a period of from 1 to 7 days; or
administration of the cannabinoid is to be carried out after a recovery phase lasting from 1 to 7 days, during which neither the chemotherapeutic agent nor the cannabinoid is administered.

23. The pharmaceutical composition for use according to claim 22, wherein cyclic administration of the cannabinoid is to be undertaken for at least 6 months.

24. The pharmaceutical composition for use according to claim 16, wherein administration of the cannabinoid is sublingual.
